(19) United States
(12) Patent Application Publication
(10) Pub. No.: US 2010/0297072 A1
(43) Pub. Date: Nov. 25, 2010

(54) Combinations of Immunostimulatory Agents, Oncolytic Virus, and Additional Anticancer Therapy

(76) Inventor: Ronald A. DePinho, Brookline, MA (US)

Correspondence Address: WILLIAMS, MORGAN & AMERSON, 10333 RICHMOND, SUITE 1100, HOUSTON, TX, 77042, US

(21) Appl. No.: 12/776,488

(22) Filed: May 10, 2010

Related U.S. Application Data
(60) Provisional application No. 61/179,480, filed on May 19, 2009.

(51) Int. Cl. A61K 38/20; A61K 39/00; A61K 39/395; A61K 38/19; A61P 35/00

(52) U.S. Cl. 424/85.2; 424/184.1; 424/174.1; 424/85.1

(57) ABSTRACT

A method comprising administering, to a mammal, an oncolytic virus and an immunostimulatory agent. A method of treating a first tumor, a second tumor, or both in a mammal having a first tumor, comprising administering an oncolytic virus into the first tumor; and administering an immunostimulatory agent systemically to the mammal. A kit comprising an oncolytic virus, an immunostimulatory agent, and instructions for administering the oncolytic virus and the immunostimulatory agent to a mammal. A kit comprising an oncolytic virus, an immunostimulatory agent, and instructions for treating a second tumor in a mammal having a first tumor by administering an oncolytic virus into the first tumor; and administering an immunostimulatory agent systemically to the mammal. The methods optionally include administering an additional anticancer therapy.

This application claims priority from U.S. provisional patent application Ser. No. 61/179,480, filed on May 19, 2009, which is incorporated herein by reference.

### BACKGROUND OF THE INVENTION

The present invention relates to cancer treatments.

### SUMMARY OF THE INVENTION

In one embodiment, the present invention relates to a method comprising administering to a mammal an oncolytic virus and an immunostimulatory agent. In one embodiment, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) granulocyte-macrophage colony stimulating factor (GM-CSF); and two or more thereof.

In one embodiment, the present invention relates to a method of treating a first tumor, a second tumor, or both in a mammal having a first tumor. The method comprises administering an oncolytic virus into the first tumor; and administering an immunostimulatory agent systemically to the mammal. In one embodiment, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

In another embodiment, the present invention relates to a kit comprising an oncolytic virus, an immunostimulatory agent, and instructions for administering the oncolytic virus and the immunostimulatory agent to a mammal.

### DESCRIPTION OF ILLUSTRATIVE EMBODIMENTS

In one embodiment, the present invention relates to a method comprising administering, to a mammal having a tumor, an oncolytic virus and an immunostimulatory agent. In a further embodiment, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

In one embodiment, the oncolytic virus is selected from the group consisting of paramyxovirus, reovirus, herpesvirus, adenovirus, and Semliki Forest virus. In one further embodiment, the paramyxovirus is selected from the group consisting of Newcastle Disease Virus (NDV), measles virus, and mumps virus. In one yet further embodiment, the NDV is from a strain selected from the group consisting of MTH68/H, PV-701, and 73-T. Oncolytic viruses, including specific ones enumerated above, will be described in more detail below.

As used herein, the term “multiplicity of infection” or “MOI” refers to the ratio of infectious virus particles to tumor cells.

An “isolated” or “purified” virus is substantially free of cellular or other contaminating material from a cell culture or other medium in which the virus is propagated. (Water, aqueous solutions, and materials known in the art for use in storing or administering viruses, are not contaminating material). “Substantially free” refers to preparations of the virus having less than about 50% by weight of contaminating material, such as less than about 40%, less than about 30%, less than about 20%, less than about 10%, less than about 5%, less than about 2.5%, less than about 1%, less than about 0.5%, or less than about 0.1% by weight of contaminating material.

An oncolytic virus, as used herein, is a virus that is able to infect and lyse cancer cells. Replication of an oncolytic virus can both facilitate tumor cell destruction and produce dose amplification at the tumor.

NDV is a negative-sense single-stranded RNA virus that causes a highly contagious disease affecting several domesticated and wild avian species. Specifically, NDV is classified as an avian paramyxovirus-1 (APMV-1) in the rubulavirus genus of the family paramyxoviridae in the order mononegaviralis. NDV is an enveloped virus of 100-300 nm diameter with a negative-sense single stranded RNA genome of roughly 16,000 nucleotides. The NDV genome contains 6 genes encoding 6 major polypeptides: L, HN, F, M, P and NP. The RNA dependent RNA polymerase involves the proteins L, P and NP which are translated in infected cells at free ribosomes in the cytoplasm. The F glycoprotein is synthesized as an inactive precursor (F0, 67 kDa), which undergoes proteolytic cleavage to yield the biologically active protein consisting of the disulfide-linked chains F1 (55 kDa) and F2 (12.5 kDa).

Various field-isolated forms of NDV have been classified according to their virulence: velogenic (highly pathogenic), mesogenic (intermediately pathogenic), or lentogenic (apathogenic).

Although NDV is generally thought to pose no hazard to human health, human exposure to the virus may lead to mild conjunctivitis and flu-like symptoms (e.g., mild fever for about 24 hr). Enveloped viruses such as NDV enter the cell through two main pathways: 1) direct fusion between the envelope and the plasma membrane and 2) receptor mediated endocytosis. For NDV, it has been established that the membrane fusion process takes place at the host plasma membrane in a pH-independent manner. Activation of the fusion protein F occurs through interaction of the viral glycoproteins with the sialic acid containing cellular receptors such as gangliosides and N-glycoproteins. In addition, it was recently shown that NDV may infect cells also through a caveolae-dependent endocytic pathway as an alternative route. A certain percentage of virions might become endocytosed to endosomes where fusion would occur at lowered pH. The ordered assembly and release of infectious NDV particles has been shown to depend on membrane lipid rafts. These are defined as cholesterol- and sphingolipid-rich microdomains in the exoplasmic leaflet of cellular plasma membranes. Newly produced virions showed an accumulation of HN, F and NP viral proteins in lipid rafts early after synthesis and contained the lipid raft-associated proteins caveolin-1, flotillin-2 and actin but not the non-lipid raft associated transferring receptor.

Various NDV strains have been found to be either lytic or nonlytic for human cells. Lytic strains generally produce activated hemagglutinin-neuraminidase and fusion protein molecules in the outer coat of progeny viruses in human cells, whereas nonlytic strains generally produce inactive versions of these molecules. Though not to be bound by theory, entry of NDV into a human cell may depend on activated hemagglutinin-neuraminidase and fusion protein molecules on the viral surface binding to sialic acid-containing molecules on the surface of a human cell.

Lytic and nonlytic strains of NDV also differ in the mechanisms by which they kill infected cells. Among lytic strains, the budding of progeny viruses containing activated hemagglutinin-neuraminidase and fusion protein molecules in their outer coats generally causes the plasma membrane of NDV-infected cells to fuse with the plasma membrane of adjacent cells, leading to the production of large, inviable fused cells (syncytia). Nonlytic strains kill infected cells more slowly, generally by viral disruption of normal host cell metabolism. The progeny virus particles made by non-lytic strains contain inactive versions of F molecules.

NDV exerts oncolysis by both intrinsic and extrinsic caspase-dependent pathways of cell death. Oncolytic NDV strains are cytotoxic to human tumor cell lines of ecto-, endo, and mesodermal origin. Such cytotoxicity is primarily due to multiple caspase dependent pathways of apoptosis. NDV triggers apoptosis by activating the mitochondrial/intrinsic pathway of apoptosis. NDV infection results in a loss of mitochondrial membrane potential and a release of mitochondrial protein cytochrome C. In addition, NDV infection leads to early activation of caspase 9 and caspase 3. In contrast, cleavage of caspase 8, which is predominantly activated by the death receptor pathway, is a TRAIL induced late event in NDV mediated apoptosis of tumor cells. The death signals generated by NDV in tumor cells ultimately converge at the mitchondria.

Either type of NDV strain generally replicates much more readily in human cancer cells than in most human cells. Certain NDV strains can replicate up to 10,000 times better in human neoplastically transformed cells than in most human cells. For example, while in non-tumorigenic human peripheral blood mononuclear cells (PBMC) the replication cycle of NDV is stopped after production of positive strand RNA, PBMC tumor cells continue in the replication cycle and copy viral genomes within 10-50 hours after infection.

Exemplary NDV strains include 73-T, MTH-68, Ulster, and NDV-HUJ. Sinkovics, et al., J. Clin. Virol. 16:1-15 (2000); Freeman, et al., Molec. Therapy 13:221 (2006); U.S. Pat. No. 7,223,389; WO 2005/051330; WO 2005/051433; Csatary, et al., Anticancer Res. 19:635-638 (1999); Csatary, et al., J. Am. Med. Assoc. 281:1588-1589 (1999); Csatary, et al., J. Neurooncol. 67:83-93 (2004).

Other oncolytic viruses include Herpes virus, Reovirus, E1B deleted adenovirus, Vesicular Stomatitis Virus, and Pox viruses. These oncolytic viruses have the potential to not only destroy tumor cells, but also release antigens from the destroyed tumor cells, thereby triggering an immune response.

As discussed above, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

In one embodiment, the immunostimulatory agent is a CTLA-4 blocking agent that specifically binds to the extracellular domain of CTLA-4 and blocks the binding of CTLA-4 to CD80 or CD86.

In one embodiment, the CTLA-4 blocking agent comprises an antibody or an antigen-binding fragment thereof. CTLA-4 blocking agents, including those comprising an antibody or an antigen-binding fragment thereof, will be described in more detail below and in U.S. Pat. No. 7,229,628, hereby incorporated herein by reference.

Granulocyte-macrophage colony stimulating factor (GM-CSF) stimulates stem cells to produce granulocytes and monocytes (proto-macrophages) and encourages dendritic cell development. The GM-CSF can be derived from any source, conveniently by isolation from expression in a recombinant microorganism transformed with a coding region encoding GM-CSF. In one embodiment, the GM-CSF is at least 95% identical (such as at least 96%, 97%, 98%, 99%, 99.5%, 99.9%, or 100% identical) with the native GM-CSF of the mammal to which the immunostimulatory agent is to be administered.

In one embodiment, the GM-CSF is derived from a recombinant strain of the oncolytic virus referred to above. In one embodiment, the coding region encoding the GM-CSF in the recombinant oncolytic virus is placed under the control of a constitutive promoter or a promoter induced by conditions prevailing after injection of the oncolytic virus into a tumor cell in vivo. Though not to be bound by theory, a recombinant oncolytic virus expressing GM-CSF may produce an enhanced dendritic cell effect (discussed below). Toll-like receptor (TLR) agonists in the recombinant oncolytic virus may then activate the dendritic cells, to then activate antigen-specific T-cells.

Though not to be bound by theory, a CTLA-4 blocking agent is expected to release T cells from inhibitory signals mediated through CTLA-4. CTLA-4 mediated signals apparently inhibit cell cycle progression and IL-2 expression. The T cell response to antigen and co-stimulatory CD28 signaling is thereby upregulated in the presence of CTLA-4 blocking agents. The CTLA-4 blocking agents do not promote a generalized proliferation of unstimulated T cells.

In vivo T cell mediated responses include the generation of cytolytic T cells, and the majority of antibody responses, particularly those involving class switching of immunoglobulin isotypes. The antigenic stimulus may be the presence of viral antigens on infected cells; tumor cells that express proteins or combinations of proteins in an unnatural context or generated in the course of somatic mutation of genes encoding such proteins; parasitic or bacterial infection; or an immunization, e.g. vaccination with tumor antigens, etc. In vitro, the subject methods may be used to increase the response of cultured T cells to antigen. Such activated T cells find use in adoptive immunotherapy, to study the mechanisms of activation, in drug screening, etc.

CTLA-4 blocking agents are molecules that specifically bind to the extracellular domain of CTLA-4 protein, and block the binding of CTLA-4 to its counter-receptors, e.g. CD80, CD86, etc. Usually, the binding affinity of the blocking agent will be at least about 100 μM. The blocking agent will be substantially unreactive with related molecules to CTLA-4, such as CD28 and other members of the immunoglobulin superfamily. Molecules such as CD80 and CD86 are therefore excluded as blocking agents. Further, blocking agents do not activate CTLA-4 signaling. Conveniently, this is achieved by the use of monovalent or bivalent binding molecules. It will be understood by one of skill in the art that the following discussions of cross-reactivity and competition between different molecules is intended to refer to molecules having the same species of origin, e.g. human CTLA-4 binds human CD80 and 86, etc.

Candidate blocking agents may be screened for their ability to meet this criteria; this screening is a matter of routine experimentation for the person of ordinary skill in the art. For example, assays to determine affinity and specificity of binding are known in the art, including competitive and non-competitive assays. Assays of interest include ELISA, RIA, flow cytometry, etc. Binding assays may use purified or semi-purified CTLA-4 protein, or alternatively may use T cells that express CTLA-4, e.g. cells transfected with an expression construct for CTLA-4; T cells that have been stimulated through cross-linking of CD3 and CD28; the addition of irradiated allogeneic cells, etc. As an example of a binding assay, purified CTLA-4 protein is bound to an insoluble support, e.g. microtiter plate, magnetic beads, etc. The candidate blocking agent and soluble, labeled CD80 or CD86 may be added to the cells, and the unbound components may be then washed off. The ability of the blocking agent to compete with CD80 and CD86 for CTLA-4 binding is determined by quantitation of bound, labeled CD80 or CD86. Confirmation that the blocking agent does not cross-react with CD28 may be performed with a similar assay, substituting CD28 for CTLA-4. Suitable molecules will have at least about 10³ less binding to CD28 than to CTLA-4, more usually at least about 10⁴ less binding.

Generally, a soluble monovalent or bivalent binding molecule will not activate CTLA-4 signaling. A functional assay that detects T cell activation may be used for confirmation. For example, a population of T cells may be stimulated with irradiated allogeneic cells expressing CD80 or CD86, in the presence or absence of the candidate blocking agent. An agent that blocks CTLA-4 signaling will cause an increase in the T cell activation, as measured by proliferation and cell cycle progression, release of IL-2, upregulation of CD25 and CD69, etc. It will be understood by one of skill in the art that expression on the surface of a cell, packaging in a liposome, adherence to a particle or well, etc. will increase the effective valency of a molecule.

Blocking agents include peptides, small organic molecules, peptidomimetics, soluble T cell receptors, antibodies, or the like. Antibodies are a preferred blocking agent. Antibodies may be polyclonal or monoclonal; intact or truncated, e.g. F(ab′)₂, Fab, Fv; xenogeneic, allogeneic, syngeneic, or modified forms thereof, e.g. humanized, chimeric, etc.

In many cases, the blocking agent will be an oligopeptide, e.g. antibody or fragment thereof, etc., but other molecules that provide relatively high specificity and affinity may also be employed. Combinatorial libraries provide compounds other than oligopeptides that have the necessary binding characteristics. Generally, the affinity will be at least about 10⁻⁶, more usually about 10⁻⁸ M, i.e. binding affinities normally observed with specific monoclonal antibodies.

A number of screening assays are available for blocking agents. The components of such assays will typically include CTLA-4 protein, and optionally a CTLA-4 activating agent, e.g. CD80, CD86, etc. Generally, a plurality of assay mixtures may be run in parallel with different agent concentrations to obtain a differential response to the various concentrations. Typically, one of these concentrations serves as a negative control, i.e. at zero concentration or below the level of detection.

Conveniently, in these assays one or more of the molecules will be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

One screening assay of interest is directed to agents that interfere with the activation of CTLA-4 by its counter-receptors. Quantifying activation may achieved by a number of methods known in the art. For example, the inhibition of T cell activation may be determined by quantifying cell proliferation, release of cytokines, etc.

Other assays of interest are directed to agents that block the binding of CTLA-4 to its counter-receptors. The assay mixture will comprise at least a portion of the natural counter-receptor, or an oligopeptide that shares sufficient sequence similarity to provide specific binding, and the candidate pharmacological agent. The oligopeptide may be of any length amenable to the assay conditions and requirements, usually at least about 8 aa in length, and up to the full-length protein or fusion thereof. The CTLA-4 may be bound to an insoluble substrate. The substrate may be made in a wide variety of materials and shapes e.g. microtiter plate, microbead, dipstick, resin particle, etc. The substrate is chosen to minimize background and maximize signal to noise ratio. Binding may be quantitated by a variety of methods known in the art. After an incubation period sufficient to allow the binding to reach equilibrium, the insoluble support is washed, and the remaining label quantitated. Agents that interfere with binding will decrease the detected label.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group, preferably at least two of the functional chemical groups. The candidate agents often comprise cyclical carbon or heterocyclic structures and/or aromatic or polyaromatic structures substituted with one or more of the above functional groups. Candidate agents may be also found among biomolecules including peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs.

A variety of other reagents may be included in the screening assay. These include reagents like salts, neutral proteins, e.g. albumin, detergents, etc which may be used to facilitate optimal protein-DNA binding and/or reduce non-specific or background interactions. Also reagents that otherwise improve the efficiency of the assay, such as protease inhibitors, nuclease inhibitors, anti-microbial agents, etc. may be used.

Suitable antibodies for use as blocking agents may be obtained by immunizing a host animal with peptides comprising all or a portion of CTLA-4 protein. Suitable host animals include mouse, rat, sheep, goat, hamster, rabbit, etc. The origin of the protein immunogen may be mouse, human, rat, monkey etc. The host animal will generally be a different species than the immunogen, e.g. mouse CTLA-4 used to immunize hamsters, human CTLA-4 to immunize mice, etc. The human and mouse CTLA-4 contain highly conserved stretches in the extracellular domain (Harper et al. (1991) J. Immunol. 147:1037-1044). Peptides derived from such highly conserved regions may be used as immunogens to generate cross-specific antibodies.

The immunogen may comprise the complete protein, or fragments and derivatives thereof Preferred immunogens comprise all or a part of the extracellular domain of human CTLA-4 (e.g., amino acid residues 38-161), where these residues contain the post-translation modifications, such as glycosylation, found on the native CTLA-4. Immunogens comprising the extracellular domain may be produced in a variety of ways known in the art, e.g. expression of cloned genes using conventional recombinant methods, isolation from T cells, sorted cell populations expressing high levels of CTLA-4, etc.

Where expression of a recombinant or modified protein is desired, a vector encoding the desired portion of CTLA-4 will be used. Generally, an expression vector will be designed so that the extracellular domain of the CTLA-4 molecule is on the surface of a transfected cell, or alternatively, the extracellular domain is secreted from the cell. When the extracellular domain is to be secreted, the coding sequence for the extracellular domain will be fused, in frame, with sequences that permit secretion, including a signal peptide. Signal peptides may be exogenous or native. A fusion protein of interest for immunization joins the CTLA-4 extracellular domain to the constant region of an immunoglobulin. For example, a fusion protein comprising the extracellular domain of mouse CTLA-4 joined to the hinge region of human Cg1 (e.g., hinge-CH2-CH3) domain may be used to immunize hamsters.

When the CTLA-4 is to be expressed on the surface of the cell, the coding sequence for the extracellular domain will be fused, in frame, with sequences encoding a peptide that anchors the extracellular domain into the membrane and a signal sequence. Such anchor sequences include the native CTLA-4 transmembrane domain, or transmembrane domains from other cell surface proteins, e.g. CD4, CD8, sIg, etc. Mouse cells transfected with the human CTLA-4 gene may be used to immunize mice and generate antibodies specific for the human CTLA-4 protein.

Monoclonal antibodies may be produced by conventional techniques. Generally, the spleen and/or lymph nodes of an immunized host animal provide a source of plasma cells. The plasma cells may be immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatant or ascites fluid by conventional techniques, e.g. affinity chromatography using CTLA-4 bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain may be ligated to a spacer encoding at least about 4 amino acids of small neutral amino acids, including glycine and/or serine. The protein encoded by this fusion allows assembly of a functional variable region that retains the specificity and affinity of the original antibody.

For in vivo use, particularly for injection into humans, it is desirable to decrease the antigenicity of the blocking agent. An immune response of a recipient against the blocking agent will potentially decrease the period of time that the therapy is effective. Methods of humanizing antibodies are known in the art. The humanized antibody may be the product of an animal having transgenic human immunoglobulin constant region genes (see, for example, PCT Publications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g. by protease or chemical cleavage. Alternatively, a truncated gene may be designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

Situations characterized by a deficient host T cell response to antigen include chronic infections, tumors, immunization with peptide vaccines, and the like. Administration of the subject CTLA-4 blockers to such hosts specifically changes the phenotype of activated T cells, resulting in increased response to antigen mediated activation.

The CTLA-4 blocking agent is administered at a dose effective to increase the response of T cells to antigenic stimulation. The response of activated T cells may be affected by the subject treatment to a greater extent than resting T cells. The determination of the T cell response may vary with the condition that is being treated. Useful measures of T cell activity may be proliferation, the release of cytokines, e.g. IL-2, IFNg, TNFa; T cell expression of markers such as CD25 and CD69; and other measures of T cell activity as known in the art.

Commercially available CTLA-4 blocking agents include ipilimumab (Bristol-Myers Squibb, New York, N.Y.) and tremilimumab (Pfizer, New York, N.Y.).

In one embodiment, the immunostimulatory agent is interleukin-21 (IL-21). IL-21 has been described by Parrish-Novak, et al., Nature 408:57-63 (2000); Wang, et al., Cancer Res. 63:9016-9022 (2003); and Thompson, et al., J. Clin. Oncol. 26:2034-2039 (2008). These documents are hereby incorporated herein by reference in their entirety.

Interleukin-21 (IL-21) is a class I cytokine that affects both innate and adaptive immunity. Effects of IL-21 include activation, increased proliferation, and prolonged survival of tumor-specific CD8 cytotoxic T lymphocytes; enhancement of T-cell dependent B cell proliferation and antibody production; and terminal differentiation and activation of natural killer cells. Unlike IL-2, IL-21 renders CD4 T cells resistant to regulatory T cell suppression and does not enhance proliferation of regulatory T cells; IL-21 may also lead to enhanced generation of memory T cells. IL-21 has been reported to have antitumor effects in various preclinical cancer models.

In one study, established subcutaneous tumor in mice was treated by systemically administering plasmid DNA encoding murine IL-21 using a hydrodynamics-based gene delivery technique. Administration of IL-21 plasmid DNA resulted in high levels of circulating IL-21 in vivo. Treatment of tumor-bearing mice with IL-21 plasmid DNA significantly inhibited the growth of B16 melanoma and MCA205 fibrosarcoma in a dose-dependent manner without significant toxicity and increased the survival rate, compared with mice treated with control plasmid DNA. In vivo depletion of either CD4_{—} or CD8_T cells did not affect IL-21-mediated antitumor activity. However, depletion of NK cells substantially abolished IL-21-induced tumor inhibition. Consistent with this, the antitumor activity of IL-21 seemed to be mediated through enhanced cytolytic activity of NK cells. The study suggested that IL-21 has significant antitumor activity and may have therapeutic potential as an antitumor agent in the clinic.

In one embodiment, the immunostimulatory agent is anti-CD40. CD40 is a member of the TNF superfamily and is expressed on B cells and dendritic cells. CD40 ligand is expressed on activated T-cells. Stimulation of CD40 on dendritic cells induces dendritic cell activation and release of IL-12. Stimulatory antibodies against CD40 can enhance antigen-specific immune responses.

Administration of the immunostimulatory agent may be combined with administration of cytokines that stimulate antigen presenting cells, e.g., granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), etc. Additional proteins and/or cytokines known to enhance T cell proliferation and secretion, such as IL-1, IL-2, B7, anti-CD3 and anti-CD28 can be employed simultaneously or sequentially with the immunostimulatory agent to augment the immune response. Administration of the immunostimulatory agent may be combined with the transfection of tumor cells or tumor-infiltrating lymphocytes with genes encoding for various cytokines or cell surface receptors (see Ogasawara et al. (1993) Cancer Res. 53:3561-8; and Townsend et al. (1993) Science 259:368-370). For example, it has been shown that transfection of tumor cells with cDNA encoding CD80 leads to rejection of transfected tumor cells, and can induce immunity to a subsequent challenge by the non-transfected parent tumor cells (Townsend et al. (1994) Cancer Res. 54:6477-6483).

Tumor-specific host T cells may be combined ex vivo with the immunostimulatory agent and tumor antigens or cells, and reinfused into the patient. When administered to a host, the stimulated cells induce a tumoricidal reaction resulting in tumor regression. The host cells may be isolated from a variety of sources, such as lymph nodes, e.g. inguinal, mesenteric, superficial distal auxiliary, etc.; bone marrow; spleen; or peripheral blood, as well as from the tumor, e.g. tumor infiltrating lymphocytes. The cells may be allogeneic or, preferably, autologous. For ex vivo stimulation, the host cells may be aseptically removed, and may be suspended in any suitable media, as known in the art. The cells may be stimulated by any of a variety of protocols, particularly combinations of B7, anti-CD28, etc., in combination with the blocking agents. The stimulated cells may be reintroduced to the host by injection, e.g. intravenous, intraperitoneal, etc. in a variety of pharmaceutical formulations, including such additives as binder, fillers, carriers, preservatives, stabilizing agents, emulsifiers and buffers. Suitable diluents and excipients include water, saline, glucose and the like.

Tumor cells whose growth may be decreased by administration of an immunostimulatory blocking agent include carcinomas e.g. adenocarcinomas, which may have a primary tumor site in the breast, ovary, endometrium, cervix, colon, lung, pancreas, eosophagus, prostate, small bowel, rectum, uterus or stomach; and squamous cell carcinomas, which may have a primary site in the lungs, oral cavity, tongue, larynx, eosophagus, skin, bladder, cervix, eyelid, conjunctiva, vagina, etc. Other classes of tumors that may be treated include sarcomas, e.g. myogenic sarcomas; neuromas; melanomas; leukemias, certain lymphomas, trophoblastic and germ cell tumors; neuroendocrine and neuroectodermal tumors.

Tumors of particular interest include those that present tumor-specific antigens. Such antigens may be present in an abnormal context, at unusually high levels, or may be mutated forms. The tumor antigen may be administered with the subject blocking agents to increase the host T cell response against the tumor cells. Such antigen preparations may comprise purified protein, or lysates from tumor cells.

Examples of tumor antigens include cytokeratins, particularly cytokeratin 8, 18 and 19, as an antigen for carcinomas. Epithelial membrane antigen (EMA), human embryonic antigen (HEA-125); human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. Chromagranin-A and synaptophysin are antigens of neuroendocrine and neuroectodermal tumors. Of particular interest may be aggressive tumors that form solid tumor masses having necrotic areas. The lysis of such necrotic cells is a rich source of antigens for antigen-presenting cells.

Administration of an immunostimulatory agent may be contra-indicated for certain lymphomas. In particular, T cell lymphomas may not benefit from increased activation. CD80 antigen is strongly expressed by the Reed-Sternberg cells in Hodgkin's disease, which are frequently surrounded by CD28-expressing T cells (Delabie et al. (1993) Blood 82:2845-52). It has been suggested that the accessory cell function of Reed-Sternberg cells leads to T cell activation, and contributes to the Hodgkin's syndrome.

Many conventional cancer therapies, such as chemotherapy and radiation therapy, severely reduce lymphocyte populations. While administration of an immunostimulatory agent may alleviate this immunosuppression to some extent, one course of combined treatment may use such lymphotoxic therapies before and/or after the subject therapy in order to further release tumor antigens or decrease immunoregulatory lymphocyte populations.

Adjuvants potentiate the immune response to an antigen. The immunostimulatory agent is used as an adjuvant to increase the activation of T cells, and to increase the class switching of antibody producing cells, thereby increasing the concentration of IgG class antibodies produced in response to the immunogen. The immunostimulatory agent is combined with an immunogen in a physiologically acceptable medium, in accordance with conventional techniques for employing adjuvants. The immunogen may be combined in a single formulation with the immunostimulatory agent, or may be administered separately. Immunogens include polysaccharides, proteins, protein fragments, haptens, etc. Of particular interest is the use with peptide immunogens. Peptide immunogens may include tumor antigens and viral antigens or fragments thereof, as described above.

The immunostimulatory agent can be used during the immunization of laboratory animals, e.g. mice, rats, hamsters, rabbits, etc. for monoclonal antibody production. Administration of the immunostimulatory agent increases the level of response to the antigen, and increases the proportion of plasma cells that undergo class switching.

The immunostimulatory agent can be administered in vitro to increase the activation of T cells in culture, including any in vitro cell culture system, e.g. immortalized cell lines, primary cultures of mixed or purified cell populations, non-transformed cells, etc. Of particular interest are primary T cell cultures, where the cells may be removed from a patient or allogeneic donor, stimulated ex vivo, and reinfused into the patient.

In some cases it may be desirable to limit the period of treatment due to excessive T cell proliferation. The limitations may be empirically determined, depending on the response of the patient to therapy, the number of T cells in the patient, etc. The number of T cells may be monitored in a patient by methods known in the art, including staining with T cell specific antibodies and flow cytometry.

The functional effect of immunostimulation may also be induced by the administration of other agents that mimic the change in intra-cellular signaling observed with the subject invention. For example, it is known that specific cytoplasmic kinases may be activated in response to binding of extracellular receptors. Agents that block the kinase activity would have a similar physiological effect as blocking receptor binding. Similarly, agents that increase cyclic AMP, GTP concentrations and intracellular calcium levels can produce physiological effects that are analogous to those observed with extracellular receptor binding.

Any mammal having a tumor may receive the oncolytic virus and the immunostimulatory agent. The mammal may be man or a mammal having economic or esthetic utility for man, e.g., farm animals, service animals, or pets. In one embodiment, the mammal is selected from the group consisting of man, non-human primates, ovines, bovines, equines, porcines, canines, felines, mice, and rats.

In one embodiment, the tumor is in an organ selected from the group consisting of brain, lung, skin, mouth, esophagus, stomach, small intestine, large intestine, colon, liver, kidney, breast, ovary, prostate gland, testicle, pancreas, bladder, and lymph node.

The oncolytic virus and the immunostimulatory agent may be administered to the mammal via the same route or via different routes. In one embodiment, the oncolytic virus is administered into the tumor, for example, by intratumoral injection, and the immunostimulatory agent is administered systemically, for example, intravascularly, subcutaneously, peritoneally, etc.

Alternative routes for administering the oncolytic virus include intravenous injection, intramuscular injection, inhalation (which may be particularly suitable for tumors of the lung), or rectal (which may be particularly suitable for tumors of the large intestine or colon).

Dosage levels of the oncolytic virus may be routinely selected by a physician or veterinarian. Desirably, the dosage of the oncolytic virus is large enough to rapidly bring about a desired therapeutic response, but small enough to not be toxic to the patient. (“Toxic to the patient” here includes typical symptoms of drug- or radiation-based anticancer therapies, such as severe nausea and hair loss, as well as typical symptoms of NDV infection of birds, such as severe respiratory disease, severe digestive-tract lesions, neurological damage, and symptoms of overdose of NDV to humans, such as dyspnea, diarrhea, dehydration, transient thrombocytopenia, and diffuse vascular leak. Mild fever, conjunctivitis, and other transient flu-like symptoms are not “toxic to the patient”). For example, NDV strain PV-701 is well tolerated in patients with advanced solid cancers in doses of at least 3×10⁹ infectious particles by the i.v. route and of at least 4×10¹² by the intra-tumoral route. When patients were desensitized with a lower initial dose, the maximum tolerated dose (MTD) was increased about 10-fold.

In one embodiment, the oncolytic virus is administered by intratumoral injection once per week for about two to four months, followed by a maintenance regimen of intratumoral injection once about every two to four months.

The dosage of the immunostimulatory agent may vary widely, depending upon the nature of the disease, the frequency of administration, the manner of administration, the purpose of the administration, the clearance of the agent from the host, and the like. The dosage administered will vary depending on known factors, such as the pharmacodynamic characteristics of the particular agent, mode and route of administration, age, health and weight of the recipient, nature and extent of symptoms, concurrent treatments, frequency of treatment and effect desired. The dose may be administered as infrequently as weekly or biweekly, or fractionated into smaller doses and administered daily, semi-weekly, etc. to maintain an effective dosage level. Generally, a daily dosage of active ingredient can be about 0.1 to 100 mg/kg of body weight. Dosage forms suitable for internal administration generally contain from about 0.1 mg to 500 mgs of active ingredient per unit. The active ingredient may vary from 0.5 to 95% by weight based on the total weight of the immunostimulatory agent.

Generally, the immunostimulatory agent, when administered according to a method of the present invention, can be efficacious in a lower dose than is typically observed for the immunostimulatory agent when administered without an oncolytic virus.

In one embodiment, the immunostimulatory agent is administered by intravenous injection in a dosage of about 10 mg/kg of body weight once about every two to four weeks for about two to four months, followed by a maintenance regimen of intravenous injection in a dosage of about 10 mg/kg of body weight once about every two to four months.

The subject immunostimulatory agents can be prepared as formulations at an effective dose in pharmaceutically acceptable media, for example normal saline, vegetable oils, mineral oil, PBS, etc. Therapeutic preparations may include physiologically tolerable liquids, gel or solid carriers, diluents, adjuvants and excipients. Additives may include bactericidal agents, additives that maintain isotonicity, e.g. NaCl, mannitol; and chemical stability, e.g. buffers and preservatives, or the like. The immunostimulatory agent may be administered as a cocktail, or as a single agent. For parenteral administration, the immunostimulatory agent may be formulated as a solution, suspension, emulsion or lyophilized powder in association with a pharmaceutically acceptable parenteral vehicle. Liposomes or non-aqueous vehicles, such as fixed oils, may also be used. The formulation may be sterilized by techniques known in the art.

The oncolytic virus may be administered to the mammal at a time before the immunostimulatory agent is administered. In one embodiment, the oncolytic virus is administered from 1 day to 5 days before the immunostimulatory agent is administered. Though not to be bound by theory, the oncolytic virus may play two roles. First, it may kill some tumor cells directly. Second, it may stimulate the immune system by leading to lysis of tumor cells it kills; tumor cell antigens released by lysis may then be picked up by dendritic cells and stimulate T cells, and thereby promote the killing of other tumor cells by the mammal's immune system. The immunostimulatory agent may promote the latter process. For example, a CTLA-4 blocking agent may reduce CTLA-4's activity of downregulating T cells. Viruses can also directly activate innate immunity by triggering toll-like receptors (TLRs) on immune cells.

In addition to administering the oncolytic virus and the immunostimulatory agent, in various embodiments, the method may further comprise additional steps.

In one embodiment, the method further comprises administering to the mammal an anticancer agent other than the oncolytic virus and the immunostimulatory agent. Any known anticancer agent can be administered by a route, in a dosage, and in a treatment regimen known to a person of ordinary skill in the art of anticancer therapy.

In one further embodiment, the anticancer agent is selected from the group consisting of paclitaxel, doxorubicin, vincristine, actinomycin D, altretamine, asparaginase, bleomycin, busulphan, capecitabine, carboplatin, carmustine, chlorambucil, cisplatin, cyclophosphamide, cytarabine, dacarbazine, daunorubicin, epirubicin, etoposide, fludarabine, fluorouracil, gemcitabine, hydroxyurea, idarubicin, ifosfamide, irinotecan, lomustine, melphalan, mercaptopurine, methotrexate, mitomycin, mitozantrone, oxaliplatin, procarbazine, steroids, streptozocin, taxotere, tamozolomide, thioguanine, thiotepa, tomudex, topotecan, treosulfan, UFT (uracil-tegufur), vinblastine, vindesine, and two or more thereof.

In another further embodiment, the anticancer agent is selected from the group consisting of alemtuzumab, aminoglutethimide, amsacrine, anastrozole, asparaginase, bcg, bevacizumab, bicalutamide, bleomycin, bortezomib, buserelin, busulfan, campothecin, capecitabine, carboplatin, carmustine, CeaVac, cetuximab, chlorambucil, cisplatin, cladribine, clodronate, colchicine, cyclophosphamide, cyproterone, cytarabine, dacarbazine, daclizumab, dactinomycin, daunorubicin, dienestrol, diethylstilbestrol, docetaxel, doxorubicin, edrecolomab, epirubicin, epratuzumab, erlotinib, estradiol, estramustine, etoposide, everolimus, exemestane, filgrastim, fludarabine, fludrocortisone, fluorouracil, fluoxymesterone, flutamide, gemcitabine, gemtuzumab, genistein, goserelin, huJ591, hydroxyurea, ibritumomab, idarubicin, ifosfamide, IGN-101, imatinib, interferon, interleukin-2, irinotecan, ironotecan, letrozole, leucovorin, leuprolide, levamisole, lintuzumab, lomustine, MDX-210, mechlorethamine, medroxyprogesterone, megestrol, melphalan, mercaptopurine, mesna, methotrexate, mitomycin, mitotane, mitoxantrone, mitumomab, nilutamide, nocodazole, octreotide, oxaliplatin, paclitaxel, pamidronate, pentostatin, pertuzumab, plicamycin, porfimer, procarbazine, raltitrexed, rituximab, sorafinib, streptozocin, sunitinib, suramin, tamoxifen, temozolomide, temsirolimus, teniposide, testosterone, thalidomide, thioguanine, thiotepa, titanocene dichloride, topotecan, tositumomab, trastuzumab, tretinoin, tivosinib, vatalanib, vinblastine, vincristine, vindesine, vinorelbine, and two or more thereof.

In yet another further embodiment, the anticancer agent is selected from the group consisting of MDX-010; MAb, AME; ABX-EGF; EMD 72 000; apolizumab; labetuzumab; ior-t1; MDX-220; MRA; H-11 scFv; Oregovomab; huJ591 MAb, BZL; visilizumab; TriGem; TriAb; R3; MT-201; G-250, unconjugated; ACA-125; Onyvax-105; CDP-860; BrevaRex MAb; AR54; IMC-1C11; GlioMAb-H; ING-1; Anti-LCG MAbs; MT-103; KSB-303; Therex; KW-2871; Anti-HMI.24; Anti-PTHrP; 2C4 antibody; SGN-30; TRAIL-RI MAb, CAT; Prostate cancer antibody; H22xKi-4; ABX-MA1; Imuteran; Monopharm-C; AV-299; and two or more thereof.

These anticancer agents other than the oncolytic virus and the immunostimulatory agent may be categorized by their mechanism of action into, for example, the following groups: anti-metabolites/anti-cancer agents, such as pyrimidine analogs (e.g., 5-fluorouracil, floxuridine, capecitabine, gemcitabine and cytarabine) and purine analogs, folate antagonists and related inhibitors (e.g., mercaptopurine, thioguanine, pentostatin and 2-chlorodeoxyadenosine (cladribine)); antiproliferative/antimitotic agents including natural products such as vinca alkaloids (e.g., vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (e.g., L-asparaginase, which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); antiproliferative/antimitotic antimetabolites such as folic acid analogs (e.g., methotrexate); platinum coordination complexes (e.g., cisplatin, carboplatin), procarbazine, hydroxyurea, mitotane, aminoglutethimide; hormones, hormone analogs (e.g., estrogen, tamoxifen, goserelin, bicalutamide, nilutamide) and aromatase inhibitors (e.g., letrozole, anastrozole); anticoagulants (e.g., heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein) and growth factor inhibitors (e.g., vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (e.g., trastuzumab and others listed above); cell cycle inhibitors and differentiation inducers (e.g., tretinoin); mTOR inhibitors, topoisomerase inhibitors (e.g., doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (e.g., cortisone, dexamethasone, hydrocortisone, methylpednisolone, prednisone, and prenisolone); growth factor signal transduction kinase inhibitors; mitochondrial dysfunction inducers and caspase activators; chromatin disruptors.

The person of ordinary skill in the art may find, as a matter of routine experimentation, that a reduced dosage or shorter or less intensive treatment regimen of the anticancer agent other than the oncolytic virus and the immunostimulatory agent may be effective when performed as part of the present method than when the anticancer agent other than the oncolytic virus and the immunostimulatory agent is administered by itself. In a specific embodiment, the effective dose (ED₅₀) for an anticancer agent or combination of agents, when used in combination with an oncolytic virus and an immunostimulatory agent of the instant invention, is at least 2 fold less than the ED₅₀ for the anticancer agent alone, and even more preferably at 5-fold, 10-fold, or even 25-fold less. Conversely, the therapeutic index (TI) for such an anticancer agent or combination of agents when used in combination with an oncolytic virus and an immunostimulatory agent of the instant invention can be at least 2-fold greater than the TI for conventional chemotherapeutic regimen alone, and even more preferably at 5-fold, 10-fold, or even 25-fold greater.

In one embodiment, the method further comprises administering to the mammal a radiation therapy. Any known radiation source can be administered by techniques, in a dosage, and in a treatment regimen known to a person of ordinary skill in the art of radiation therapy for cancer. The person of ordinary skill in the art may find, as a matter of routine experimentation, that a reduced dosage or shorter or less intensive treatment regimen of radiation therapy may be effective when performed as part of the present method than when radiation therapy is administered by itself.

The progress of treatment resulting from performance of the present method can be routinely monitored by techniques known to the person of ordinary skill in the art, including, but not limited to, noninvasive imaging, biopsy, and analysis of blood-borne markers of tumor activity (generally correlated with tumor mass), among others.

In another embodiment, the present invention relates to a kit comprising an oncolytic virus, an immunostimulatory agent, and instructions for administering the oncolytic virus and the immunostimulatory agent to a mammal having a tumor.

The oncolytic virus and the immunostimulatory agent can be as described above.

In one embodiment, the present invention relates to a method of treating a first tumor, a second tumor, or both in a mammal having a first tumor. The method comprises administering an oncolytic virus into the first tumor, and administering an immunostimulatory agent systemically to the mammal.

A common cancer progression is for a primary tumor to arise in a particular tissue, organ, or organ system of a mammalian body. Subsequently, one or more metastases of the primary tumor may arise in other particular tissues, organs, or organ systems of the mammalian body, typically by migration of one or more cells of the primary tumor through the mammal's blood or lymph.

Frequently, it is more difficult to treat a metastatic cancer than a primary tumor. A metastatic cancer may occur at one or more locations unamenable to various treatment options, and/or in such a profusion of sites that various treatment options may be relatively ineffective. In addition, a metastatic cancer often arises when a primary tumor is relatively advanced and the patient's prognosis is already relatively poor.

Unexpectedly, we have found that administering an oncolytic virus into a first tumor (which may be a primary tumor or a metastatic tumor) and administering an immunostimulatory agent systemically to a mammal can lead to a reduction in the size of the second tumor (which may be a metastatic tumor) greater than that found when administering an oncolytic virus into the first tumor alone or administering an immunostimulatory agent systemically to the mammal alone.

The word “tumor” is used herein to any neoplastic cells, not necessarily those found in a solid neoplasm, except in particular passages where the phrase or sentence including the word would indicate to the person of ordinary skill in the art that the word refers to solid neoplasms.

The oncolytic virus can be as described above. In one embodiment, the oncolytic virus is selected from the group consisting of paramyxovirus, reovirus, herpesvirus, adenovirus, and Semliki Forest virus.

The immunostimulatory agent can be as described above. In one embodiment, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

In one embodiment, the method further comprises administering a localized anticancer therapy to the first tumor. The localized anticancer therapy can be a radiation therapy, though alternatively or in addition, other localized anticancer therapies, such as targeted therapies, including targeted chemotherapy, can be used. Unexpectedly, we have found that administering an oncolytic virus into a first tumor, administering an immunostimulatory agent systemically to a mammal, and administering a localized anticancer therapy to the first tumor can lead to a reduction in the size of the second tumor greater than that found when administering an oncolytic virus into the first tumor alone, administering an immunostimulatory agent systemically to the mammal alone, or administering a localized anticancer therapy to the first tumor alone.

Administering an oncolytic virus into a first tumor and administering an immunostimulatory agent systemically to a mammal can lead to a reduction in the size of the first tumor. Also, administering an oncolytic virus into a first tumor, administering an immunostimulatory agent systemically to a mammal, and administering a localized anticancer therapy to the first tumor can lead to a reduction in the size of the first tumor.

In one embodiment, the present invention relates to a kit comprising an oncolytic virus, an immunostimulatory agent, and instructions for treating a first tumor, a second tumor, or both in a mammal having a first tumor by administering an oncolytic virus into the first tumor and administering an immunostimulatory agent systemically to the mammal.

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### EXAMPLE

Male C57B16J mice, 8-10 weeks old, were used. The mice had an average weight of 20-22 g. The mice were divided into six study groups, each containing five or six mice per group. The experimental groups were as follows:

Group 1. Mock treatment (intratumor and intraperitoneal PBS, amputation)

Group 2. X-ray treatment

Group 3. Newcastle disease virus (NDV) treatment

Group 4. NDV treatment and X-ray treatment

Group 5. AntiCTLA4 treatment

Group 6. NDV treatment, antiCTLA-4 treatment, and X-ray treatment

The following procedures were performed:

On day 0, 300,000 tumor cells (MCA205, B16ova) in a 20 μl volume of PBS (Groups 1-6) were transplanted onto the right footpad.

Six days later, 200,000 tumor cells (MCA205, B16ova) in a 100 μl volume of PBS (Groups 1-6) were transplanted onto the left flank (i.e. contralaterally).

On day 12, the footpad tumors were clearly visible. The footpad tumors of Groups 2, 4, and 6 were irradiated with 4 Gy X-rays at a dose rate of 0.528 Gy/min. The rest of the mouse's body was lead shielded during tumor irradiation.

Immediately after X-ray treatment, the footpad tumors were injected with 0.6×10⁷ NDV strain MTH68H viral particles in 10 μl of PBS, for Groups 3, 4, and 6. Group 1 received a 10 μl PBS mock injection. NDV treatment was performed 5 times per week, once daily Monday-Friday for the duration of the experiment. From the second week of the treatment onward, 20 μl of the NDV solution (1.2×10⁸ viral particles) was injected.

One hour after the first X-ray treatment, antiCTLA-4 was given intraperitoneally (100 μg/mouse in approximately 100 μl PBS) to Groups 5 and 6. Group 1 received a 100 μl PBS mock injection. Treatments were repeated five times every 3 days.

Tumor growth for both the footpad tumor and the contralateral tumor was quantified from average tumor volume for Groups 1-6.

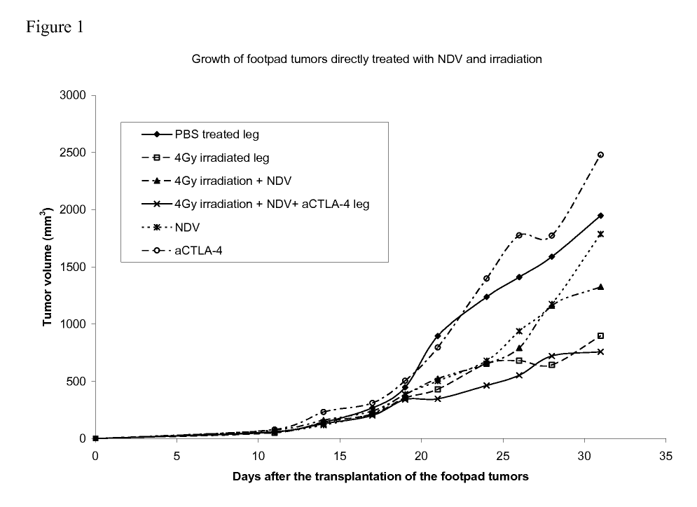

FIG. 1 shows growth of the footpad tumor for Groups 1-6. Group 6, which received a combination of irradiation, NDV, and antiCTLA-4, had a smaller average tumor volume at day 32 than all of the other groups, including both Group 2 (receiving irradiation alone) and Group 4 (receiving irradiation and NDV).

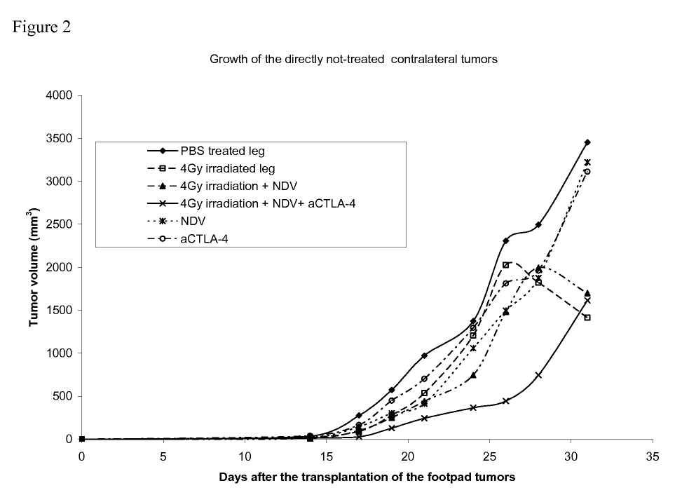

FIG. 2 shows growth of the contralateral flank tumor for Groups 1-6. As should be apparent from the description above, the flank tumor was not directly treated with irradiation, viral injection, or antiCTLA-4. Group 6, which received a combination of irradiation, NDV, and antiCTLA-4, had a smaller average tumor volume at days 19-28 than all of the other groups, including both Group 2 (receiving irradiation alone) and Group 4 (receiving irradiation and NDV).

All of the compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating a first tumor, a second tumor, or both in a mammal having a first tumor, comprising:
administering an oncolytic virus into the first tumor; and
administering an immunostimulatory agent systemically to the mammal.

2. The method of claim 1, wherein the oncolytic virus is selected from the group consisting of paramyxovirus, reovirus, herpesvirus, adenovirus, and Semliki Forest virus.

3. The method of claim 1, wherein the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

4. The method of claim 1, further comprising:
administering a localized anticancer therapy to the first tumor.

5. The method of claim 1, wherein the localized anticancer therapy is a radiation therapy.

6. A method, comprising:
administering, to a mammal, an oncolytic virus and an immunostimulatory agent.

7. The method of claim 6, wherein the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

8. The method of claim 7, wherein the immunostimulatory agent is a CTLA-4 blocking agent that specifically binds to the extracellular domain of CTLA-4 and blocks the binding of CTLA-4 to CD80 or CD86.

9. The method of claim 8, wherein the CTLA-4 blocking agent comprises an antibody or an antigen-binding fragment thereof.

10. The method of claim 6, wherein the oncolytic virus is selected from the group consisting of paramyxovirus, reovirus, herpesvirus, adenovirus, and Semliki Forest virus.

11. The method of claim 10, wherein the paramyxovirus is selected from the group consisting of Newcastle Disease Virus (NDV), measles virus, and mumps virus.

12. The method of claim 11, wherein the NDV is from a strain selected from the group consisting of MTH68/H, PV-701, and 73-T.

13. The method of claim 6, wherein the mammal has a tumor in an organ selected from the group consisting of brain, lung, skin, mouth, esophagus, stomach, small intestine, large intestine, colon, liver, kidney, breast, ovary, prostate gland, testicle, pancreas, bladder, and lymph node.

14. The method of claim 6, further comprising administering to the mammal an anticancer agent other than the oncolytic virus and the immunostimulatory agent.

15. The method of claim 14, wherein the anticancer agent is selected from the group consisting of paclitaxel, doxorubicin, vincristine, actinomycin D, altretamine, asparaginase, bleomycin, busulphan, capecitabine, carboplatin, carmustine, chlorambucil, cisplatin, cyclophosphamide, cytarabine, dacarbazine, daunorubicin, epirubicin, etoposide, fludarabine, fluorouracil, gemcitabine, hydroxyurea, idarubicin, ifosfamide, irinotecan, lomustine, melphalan, mercaptopurine, methotrexate, mitomycin, mitozantrone, oxaliplatin, procarbazine, steroids, streptozocin, taxotere, tamozolomide, thioguanine, thiotepa, tomudex, topotecan, treosulfan, UFT (uracil-tegufur), vinblastine, vindesine, and two or more thereof.

16. The method of claim 6, further comprising administering to the mammal a radiation therapy.

17. A kit, comprising:
an oncolytic virus, an immunostimulatory agent, and instructions for administering the oncolytic virus and the immunostimulatory agent to a mammal.

18. The kit of claim 17, wherein the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

19. The kit of claim 18, wherein the immunostimulatory agent is a CTLA-4 blocking agent that specifically binds to the extracellular domain of CTLA-4 and blocks the binding of CTLA-4 to CD80 or CD86.

20. The kit of claim 19, wherein the CTLA-4 blocking agent comprises an antibody or an antigen-binding fragment thereof.

21. The kit of claim 17, wherein the oncolytic virus is selected from the group consisting of paramyxovirus, reovirus, herpesvirus, adenovirus, and Semliki Forest virus.

22. The kit of claim 21, wherein the paramyxovirus is selected from the group consisting of Newcastle Disease Virus (NDV), measles virus, and mumps virus.

23. The kit of claim 22, wherein the NDV is from a strain selected from the group consisting of MTH68/H, PV-701, and 73-T.

24. A kit, comprising:
an oncolytic virus, an immunostimulatory agent, and instructions for treating a first tumor, a second tumor, or both in a mammal having a first tumor by administering an oncolytic virus into the first tumor; and administering an immunostimulatory agent systemically to the mammal.

25. The kit of claim 24, wherein the oncolytic virus is selected from the group consisting of paramyxovirus, reovirus, herpesvirus, adenovirus, and Semliki Forest virus.

26. The kit of claim 24, wherein the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) GM-CSF; and two or more thereof.

27. The kit of claim 24, wherein the instructions further comprising:
administering a localized anticancer therapy to the first tumor.

28. The kit of claim 24, wherein the localized anticancer therapy is a radiation therapy.
